(19) United States
(12) Patent Application Publication
(10) Pub. No.: US 2025/0256096 A1
(43) Pub. Date: Aug. 14, 2025

(54) SYSTEMS, DEVICES, AND METHODS FOR DELIVERING THERAPEUTIC AGENTS FOR NERVE STIMULATION

(71) Applicant: TRANQUILLUM MEDICAL, LLC, Raleigh, NC (US)

(72) Inventors: Scott MILLER, Arlington, VA (US); Christopher J. CZURA, Oyster Bay, NY (US); Marc L. ABRAMOWITZ, Aspen, CO (US)

(21) Appl. No.: 18/855,872

(22) PCT Filed: Apr. 13, 2023
(86) PCT No.: PCT/US2023/065714

§ 371 Date: Oct. 10, 2024

Related U.S. Application Data
(60) Provisional application No. 63/330,352, filed on Apr. 13, 2022.

(51) Int. Cl. A61N 1/36; A61N 1/04; A61N 1/05

(52) U.S. Cl. CPC A61N 1/36031; A61N 1/0428; A61N 1/0456; A61N 1/0551; A61N 1/36135

(57) ABSTRACT

Devices, systems, and methods for delivering therapeutic agents for nerve stimulation are used to administer a therapeutic agent to a patient, modulate one or more nerves of the patient, and optionally monitor patients' physiological parameters. The monitoring can inform the nerve modulation and/or administration of the therapeutic agent. Systems, apparatus, and methods deliver therapeutic agents for nerve stimulation. Devices administer therapeutic agents, modulate patients' nerve(s), and observe and track patient physiological parameters. Modulation can be performed during and/or after administration of a therapeutic to the patient. Targeted compounds activate peripheral nerves including via agonists of chemoreceptors or somatosensory receptors resulting in signaling in the cervical vagus nerve. These compounds demonstrate chemical nerve activation of the vagus nerve but through chemical agents engaging nerve sensors and with effects like electrical stimulation of the vagus nerve, which has therapeutic effects in multiple disease states.

### CROSS REFERENCE TO RELATED APPLICATION

This application claims benefit of priority from U.S. Provisional Application No. 63/330,352 filed on Apr. 13, 2022, the entire contents of which are incorporated by reference.

### TECHNICAL FIELD

The invention relates to novel, improved methods of therapeutic treatment and related systems and apparatus. More particularly, the invention relates to the delivery of a therapeutic agent in connection with application of nerve modulation with optional monitoring of one or more physiological parameters of a patient undergoing treatment.

### BACKGROUND

Stimulation of certain aspects of the body by implantable electrical generators has been performed for various benefits, including, for example, cardiac pacemaking, cardiac defibrillation, muscle tremor treatment, depression treatment, and modulation of effects of drug-resistant arthritis. Other approaches have used implantable electrical stimulation to address other conditions, such as pain management and opioid dependency. These efforts have focused on stimulating a nerve pathway, given nerves ability to impact movement, as well as physiological function, through their own transmission of electrical signals.

Mammals, including the human body, have a complex, multi-dimensional nervous system that regulates and controls a host of various functions important for movement, sensing, stimulation and response to various inputs, management of organ function, and other activities related to overall health and well-being. When studied, the nervous system is commonly segmented into the central nervous system, which includes the brain and spinal cord, and the peripheral nervous system, which, in turn, is further segmented into the somatic nervous system involved in signally and controlling the movement of skeletal muscles, and the autonomic nervous system which is involved in what is sometimes described as “automatic” regulatory functions.

The autonomic nervous system (ANS) has two sub-categories: the sympathetic nervous system and the parasympathetic nervous system. When in balance, these two aspects of the autonomic nervous system act in concert with other aspects of the human body to regulate a host of activities in a manner related to sustaining overall health, including, respiration, cardiovascular function, blood pressure regulation, digestion and absorption of nutrients, immune system response, inflammation response, fight or flight reactions to perceived and/or actual threats, resetting to points of relaxation and rest, and reaction to and modulation of external stimuli, such as light and temperature, among others. The balance of one's sympathetic and parasympathetic nervous systems contributes vitally to optimal health.

The nervous system functions as an important communication pathway that initiates and modulates important physiological responses to various events that can impact one's health. It reflexively engages in efferent and afferent behavior in response to various conditions. These responses assist the body's physical function in temporary circumstances, such as protective fight or flight responses. However, in other circumstances, they cause the sympathetic nervous system and the parasympathetic nervous system to shift to points of imbalance. These shifts can be due to chronic, progressed disease (such as arteriosclerosis and cardiovascular disease, autoimmune disorders), extended periods of stress, a traumatic neurological and/or physiological event, and the effects of aging over time. Remaining in these points of imbalance leads to certain immediate as well as chronic health issues if not addressed.

In connection with these responses, the body employs an increase in levels of inflammation. This inflammatory response is the body's way to try and cope with circumstances the body perceives/discerns are threatening, including conditions involving disease, sickness, injury, and invasion of the body such as through surgery or the delivery into the body of a medical implant. The body also employs inflammation in the short-term to fight off certain pathogens and other threatening elements, such as viruses and bacteria. Inflammation is also a common response to a temporary or limited injury, such as a muscle strain or bruise from an activity. Short-term, temporary increases in inflammation can be beneficial as part of a coordinated and elevated immune system response to a circumstance the body works to heal/address. However, longer-term increases in inflammation levels can be both a response to a progressing disease condition and a cause of ongoing disease progression, such as advancing cardiovascular disease or autoimmune conditions.

The body's inflammatory response must also be kept in balance. When functioning properly, the immune system sends signals to, and receives information from, the nervous system through a communication pathway sometimes called the inflammatory reflex, wherein efferent and afferent elements of the nervous system respond to potentially harmful elements, which in a healthy individual with a properly functioning immune system return (the immune system) to a previous point of balance.

Both pro-inflammatory and anti-inflammatory activity occurs through communication between the body's nervous system and its immune system. This activity occurs in part through the activation of the body's immune cells by the nervous system, which causes the release of cytokines. Cytokines are small proteins that act as cell messengers to direct the body's immune system response to a threat. When this response is appropriately proportionate to the threat to the body, the immune system action and related inflammation levels and associated cytokine response appropriately addresses the threat without significant risk over a short-term period of time. However, there are instances where the immune system can overshoot the needed response, resulting in extreme/excessive release of cytokines that are harmful and dangerous, including at high levels leading to organ damage, organ failure and even death.

Recent advances in therapeutic agents to treat certain life-threatening diseases introduce a new, external source of stress on the body which can trigger an inflammatory response. These therapeutic agents are designed to modify or otherwise change the body's immune system response in order to fight certain potentially deadly forms of disease, including forms of cancer and infectious diseases, including HIV. They can include immunotherapy agents, cell therapy agents, therapeutics for autoimmune conditions, gene therapies, MRNA-based therapeutic agents, all of which are specifically designed to modify and potentially enhance the body's ability to use the immune system to fight or prevent disease. However, a frequent, if not ubiquitous, side effect is an elevated immune system response which impacts other aspects of the body's functions in a potentially harmful way, especially with respect to the production of excessive and potentially harmful levels of cytokines. Further, certain of these therapies have as their goal the elevation of certain cytokines for their immunoprotective activity, however, they lack precise ways to manage the inherent risks associated with intentionally elevating cytokine production and its attendant cytokine release syndrome (CRS) adverse side effect.

In certain circumstances, these therapeutic agents involve removal of biologic elements from the patient or a donor, typically in the form of a blood sample or other means of extraction, to obtain and then modify or otherwise add to a component of the blood sample. The objective of this combination is to create a therapeutic agent to reintroduce into the patient that modifies aspects of the immune system's disease fighting arsenal by harnessing or supplementing the patient's immune system's attempt to fight certain disease. While a promising approach to attacking various significant diseases, such as various forms of cancer, experience has shown that modifying the immune system can result in pronounced inflammatory responses that can become more dangerous to the patient than the underlying disease being treated by the therapeutic agent.

Other forms of therapy do not use a specific blood draw, but instead attempt to genetically engineer a cellular-based response using a genetically engineered therapeutic, such as for example laboratory cultivated stem cells, gene therapies, mRNA-based approaches, or, as a further example, bispecific antibodies, which are then introduced into the patient to create an immune system response.

As a further example, an emerging form of immuno-oncology therapy known as Chimeric Antigen Receptor Therapy or CAR-T involves removing blood from a cancer patient or otherwise removing blood from a donor without cancer. This blood is processed to separate T-cells from the rest of the blood which are then tagged with one or more modifications such that the T-cells are essentially “programmed” to target a specific antigen on a tumor cell when reintroduced into the patient. This processed and programmed new therapeutic is then introduced into the cancer patient, through infusion or other such means, whereby the patient receives this CAR-T therapy using the extracted T-cells with modifications to target a form of cancer in the patient. This infusion significantly increases the number of circulating T-cells in the patient's blood stream by introducing the modified CAR-T T-cells into the patient. The intended effect of this immuno-oncology therapeutic agent is an elevated number of circulated CAR-T T-cells that are programmed to target, bind and attack identified antigen(s) on the surface of one or more cancerous tumors. CAR-T cells launch a local, therapeutic attack on specific cancer cells and use cytokines to recruit additional immune cells to attack the targeted tumor. These recruited immune cells continue to release additional cytokines, which can spill over into a systemic response involving recruitment of even more immune cells and elevated cytokine production that is not specific to the tumor and creates a dangerous systemic cytokine response syndrome (CRS) that continues to build upon itself unless checked.

The clinical results of CAR-T therapy in the short run have shown promise; however, these results have also revealed that this therapy, and other therapies that attempt to harness and/or modify the immune system, have significant and dangerous side effects. A very substantial number of patients experience a response described as turning the immune system into hyper-overdrive, such that a cytokine storm or CRS develops as an extreme inflammatory response to the CAR-T therapy. Unless the cytokine storm/CRS circumstance is addressed effectively, this extreme inflammatory response can be more deadly and result in more immediate mortality than the cancer that the patient is fighting. In certain CAR-T clinical studies, over 97 percent of treated patients experienced a CRS inflammatory response to the CAR-T therapy. Additionally, these patients also commonly experience neurological/neuro cognitive issues as an additional related side effect. Other immunotherapy approaches to treating disease, including checkpoint inhibitors, bispecific therapeutics, immune enhancement therapy, dendritic cell vaccinations, certain gene therapies, cytokine-based protein therapeutics, and other forms of cell therapy, all exhibited an immune system response expressed in dangerously elevated inflammation in the form of elevated cytokine release.

To manage a cytokine storm/cytokine release syndrome response in the context of treatment with these therapeutic agents, physicians frequently administer one or more anti-inflammatory drugs, such as a non-steroidal anti-inflammatory or another cyclooxygenase-2 inhibitor, a cytokine inhibitor (e.g., an IL-6 inhibitor IL-10 or other interleukin inhibitor, or a TNF inhibitor), anti-inflammatory steroids (corticosteroids), cytostatics such as methotrexate, or other anti-inflammatory and/or steroid drugs. The success of these anti-inflammatory and/or steroid drugs is variable, and all of these drugs when administered end up lowering the effectiveness of the immunotherapy as these drugs lower the immune system's pro-inflammatory response in order to address the cytokine storm/cytokine release syndrome (CRS) condition. Addressing the CRS concerns with current drugs involves tamping down the immune system's pro-inflammatory response. There is a linkage between the CAR-T T-Cell introduction in the body and the immune system's pro-inflammatory response, which is triggered by the CAR-T T-Cells and is key to the mechanism of action underlying the effectiveness of these therapeutic agents.

In addition to the issues associated with responding to and treating a CRS condition with current drugs, a clear early warning system/detection approach does not exist to provide an advanced and precise real-time warning to the physician of an impending cytokine storm/CRS situation, especially in the context of administering an immunotherapeutic agent to a patient. Physicians are aware of generalized risk periods (e.g., a range of 3 to 14 days for certain therapeutics), which does not support precise assessments and timely responses. To obtain more specific information, periodic patient monitoring occurs of select parameters, such as pulse rate and oxygen levels, but typically a patient blood draw must be performed to look with specificity for the presence of elevated inflammation markers indicative of cytokine storms/CRS in order for the physician to have confirmation of this condition. This approach has the drawback of essentially being a snapshot in time, which creates a double-edged set of circumstances for the patient. On the one-hand the blood draw can provide a host of specific information; however, obtaining this information takes time and because this information is derived from a specific point in time (i.e., the time of the blood draw), not over time, longitudinal inflammatory trends are not part of the information obtained and provided to the physician. Additionally, hours can pass before the blood-based data is fully compiled and presented to the attending physician, creating a potentially dangerous lag between the time of the blood draw and the delivery of the information to the physician to then determine if a dangerous cytokine storm/CRS condition is occurring with the patient. During this time, the cytokine response can escalate to a potentially dangerous point for the patient, while the physician waits for the information to determine if a clinical step is needed to address the patient's condition with an anti-inflammatory dose of medicine. Alternatively, the blood draw information may indicate a cytokine storm has not initiated, creating a potentially false point of comfort, as a cytokine storm could be in the process of initiating and escalating at a point in time after the time of the blood draw, but prior to obtaining the specific information from the blood draw.

A series of additional issues present when the physician attempts to treat the cytokine storm/CRS condition. The physician is confronted with a number of decisions, all involving notable trade-offs under current approaches, treatment therapies, and device capabilities. The physician must select a drug and a dose that has the potential to tamp down the cytokine storm/CRS condition before severe damage or death occurs to the patient, while trying to balance this step against its impact on the primary focus of trying to treat the patient's underlying condition, such as cancer in the case of a CAR-T case. The challenge for the physician is all of the anti-inflammatory drugs the physician can elect to administer have an immuno-suppressive effect, such that the physician has a difficult dilemma to navigate. The goal of the therapeutic agent delivered to the patient is to treat an underlying severe condition, and this therapeutic agent is dosed at a specific level intended to produce a therapeutic effect through a pro-inflammatory response, with the dose and related pro-inflammatory response being an amount determined to be necessary to treat a specific condition, such as a targeted cancer tumor, an infectious disease such as AIDS/HIV, autoimmune conditions including rheumatoid arthritis and inflammatory bowel disease, or other deadly or significant conditions. Therapies rely in part on the signaling of pro-inflammatory cytokines and other related immune system responses to elevate the immune system to assist with addressing a particular patient condition. Now, because of the cytokine storm/CRS situation, the physician must shift from focusing on the primary therapeutic treatment and instead shift focus to treating the potentially dangerous cytokine storm/CRS side-effect using an anti-inflammatory drug that is known to lower the performance of the therapeutic treatment targeting the primary condition, such as cancer, by tamping down the immune system's response. Current anti-inflammatory treatments to address cytokine storm/CRS side-effects use pharmacological agents that tamp down the immune system through a non-specific anti-inflammatory effect, which includes inhibiting signaling through pro-inflammatory cytokines. This means the physician must intentionally lower the benefit of the primary therapeutic treatment below the targeted effect related to the specified dosing level to effectively treat the primary condition. If the physician does not dose enough anti-inflammatory medicine to treat the cytokine storm/CRS condition, the patient can be harmed or even die from the cytokine storm/CRS condition. Additionally, the anti-inflammatory dose may not have immediate effect as it must be absorbed into the patient's circulatory system with sufficient bioavailability to impact the cytokine storm, which takes time as well, so the level of anti-inflammatory dosing, the timing of dosing, and the patient's responsiveness to the drug all impact the effectiveness of this step.

If the dosing for the cytokine storm/CRS condition is successful, the effectiveness of the primary therapeutic treatment may have been lowered to a point where the patient continues to be at risk from that condition, such as an aggressive cancer which may not be as responsive to treatment at the lower dose. The physician must find his or her way through balancing these two points, with challenging outcomes in either direction if the dosing is off relative to the CRS condition, the timing of the dose, the treatment of the patient's underlying condition that prompted the primary therapeutic treatment, and the patient's physiology and responsiveness to all of the agents and other factors. All of this happens with very limited, inconsistent information flow to the physician about the patient's evolving, dynamic condition. As mentioned earlier, blood draws/samples provide limited, time-lagged information, plus additional non-invasive monitoring capabilities are not well refined and targeted for application to cytokine storm/cytokine release syndrome situations.

An added complication with this circumstance concerns the timing of the presentation of a cytokine storm/CRS situation, which can vary by patient. For example, with certain forms of CAR-T therapy, it can be between 3 and 14 days after the infused CAR-T dose before a cytokine storm/CRS situation presents. This means the patient may not be at the hospital and/or not under close monitoring when this deadly side-effect initiates, or the patient could be under close monitoring at the hospital but still subject to a lagged response because of the timing issues for confirming the situation with blood draws and resulting blood panel reports to the attending physician. With this variable and time-lagged window, the patient may not be assessed for CRS until after pronounced symptoms are noted and the storm/CRS is underway and confirmed with the time-lagged blood draw approach mentioned above.

Clearly, a different approach is needed that preserves the therapeutic benefits of the agents while limiting, if not eliminating, the adverse and counter-productive side effects of a related cytokine storm/cytokine release syndrome event for the patient. Additionally, a better approach of monitoring the patient and informing both the patient and the attending healthcare workers, including the treating physician, is needed, including an early warning system and additional on-going, real-time, dynamic information about the patient's condition, including his or her adaptive change as therapeutic agents and other forms of treatment are administered. The current approach using intermittent information and information that is not specific to the therapy needs to be addressed in connection with providing a better alternative than immuno-suppressive anti-inflammatory medications for treating CRS.

### SUMMARY

The invention provides novel devices, systems, and methods for delivering therapeutic agents for nerve stimulation. In one example embodiment, a method of treatment comprises the steps of modulating one or more nerves of a patient, administering a therapeutic agent to the patient in need thereof, and optionally monitoring one or more physiological parameters of the patient. In methods of the invention, a monitoring step is included and informs the nerve modulation and/or the administration of the therapeutic agent. The invention also provides a related apparatus comprising a device to modulate one or more nerves of a patient before, during, or after administration of a therapeutic to the patient in need thereof and a device to monitor one or more physiological parameters of the patient. The invention also provides a related apparatus comprising a device to administer a therapeutic to the patient in need thereof. Further the invention also includes systems for delivering therapeutic agents for nerve stimulation including devices to administer therapeutic agents, devices to modulate one or more nerves of a patient, and monitoring devices to monitor physiological parameters of the patient.

### BRIEF DESCRIPTION OF THE DRAWINGS

FIG. 1 shows a schematic representation of an example embodiment of the invention, including a block diagram of a method of delivery of a therapeutic agent in connection with application of nerve modulation with optional monitoring of one or more physiological parameters of a patient undergoing treatment.

FIG. 2 shows a block diagram of an example embodiment of the invention, including nerve modulation elements of methods in accordance with the invention.

FIGS. 3A and 3B show an example of the autonomic nervous system (ANS) nerve branches in the human body.

FIG. 4 shows a block diagram of an example embodiment of the invention, including example therapeutic agents used with methods and systems in accordance with the invention.

FIG. 5 shows a block diagram of an example embodiment of the invention, including example delivery confirmation points of evidence.

FIGS. 6A-6D show cervical vagus nerve recordings obtained at baseline, after cervical applications of capsaicin at low and high concentrations, and with intradermal application, respectively, as well as cervical vagus nerve activity at baseline, after low, and after high concentrations of capsaicin were applied to the cervical vagus nerve, respectively, as well as after intradermal application of capsaicin in the forehead near trigeminal nerve endings.

FIGS. 7A-7C show cervical vagus nerve recordings obtained at baseline, after cervical applications of GSK1016790A at low and high concentrations, as well as cervical vagus nerve activity at baseline, after low, and after high concentrations of GSK1016790A were applied to the cervical vagus nerve, respectively, in the forehead near trigeminal nerve endings.

FIGS. 8A-8D show cervical vagus nerve recordings obtained at baseline, after cervical applications of WS-12 at low and high concentrations, and with intradermal application, respectively, as well as cervical vagus nerve activity at baseline, after low, and after high concentrations of WS-12 were applied to the cervical vagus nerve, respectively, as well as after intradermal application of WS-12 in the forehead near trigeminal nerve endings.

### DETAILED DESCRIPTION

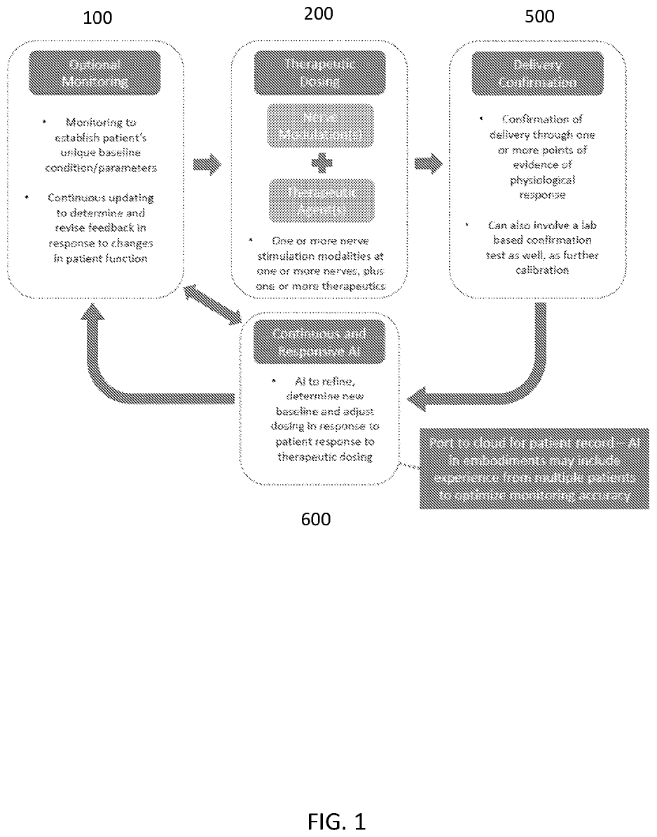

The invention provides a novel method of treatment comprising the steps of modulating one or more nerves of a patient, administering a therapeutic agent to the patient in need thereof, and optionally monitoring one or more physiological parameters of the patient. In methods of the invention a monitoring step is included and informs the nerve modulation and/or the administration of the therapeutic agent. The steps of modulating one or more nerves of a patient and administering a therapeutic agent to the patient in need thereof represent the therapeutic dosing aspects of a method of treatment according to the invention. The invention also provides a related apparatus comprising a device to modulate one or more nerves of a patient before, during or after administration of a therapeutic to the patient in need thereof and a device to monitor one or more physiological parameters of the patient. The invention also provides a related apparatus comprising a device to administer a therapeutic to the patient in need thereof. The devices can be parts of systems for delivering therapeutic agents for nerve stimulation in accordance with the invention. For example, in one example embodiment, the invention also includes systems for delivering therapeutic agents for nerve stimulation including devices to administer therapeutic agents, devices to modulate one or more nerves of a patient, and monitoring devices to monitor physiological parameters of the patient. FIG. 1 depicts the elements and identifies some features and advantages of treatment methods and apparatus according to the invention.

Modulating one or more nerves of a patient produces a physiological response that is beneficial to the patient undergoing treatment. These beneficial physiological responses include, but are not limited to, inhibiting the dissemination of pro-inflammatory cytokines, increasing heart rate variability, and altering electroencephalography (EEG)-detected brainwave activity and/or pupillary responses to be consistent with improved autonomic tone, as well as reductions in pain. A method of the invention combines nerve modulation with administering one or more therapeutic agents to treat a patient in need thereof. The step of modulating one or more nerves of the patient may be done before, during or after administering one or more therapeutic agents to the patient. Methods of treatment according to the invention have increased therapeutic efficacy over administering therapeutic agents without nerve modulation. With this increased therapeutic efficacy methods of treatment according to the invention in some embodiments allow for treatment with lower doses of the therapeutic agent(s). In addition, administration of anti-inflammatory drugs to quell the cytokine response syndrome that may spill into the systemic circulation will also diffuse into the tumor microenvironment and will inhibit the activity of the infused engineered (such as CART-T, CAR-M (Macrophages), CAR-N (Nutrifiles), autologous immune enhancement therapies and cell-based vaccines or other) cells. Neuromodulation therapies, including vagus nerve stimulation (VNS) or trigeminal nerve stimulation (TNS), have the distinct benefit of acting on circulating immune cells that travel through the blood stream to the spleen, which receive the vagus nerve signal induced by VNS/TNS, or through direct splenic, liver, or other organ stimulation. The infused therapeutic engineered cells, once administered, migrate to the condition they have been programmed to treat, such as a tumor they have been programmed to attack, and remain resident with that tumor. As a result, they do not receive the VNS/TNS signal, and are not shifted into an anti-inflammatory state, and thus retain their anti-cancer properties and related pro-inflammatory signaling at a targeted point. This is in contrast to an administered anti-inflammatory pharmacologic agent, which will diffuse in a non-selective, systemic manner, including into the targeted treatment area, such as the tumor and reduce the tumor-fighting capabilities of the CAR-T cells, or other therapeutic agents.

Nerve modulation through implantable devices that employ electrical stimulation has also been shown to improve incontinence when applied to the sacral nerve of patients and possibly the tibial nerve of patients. However, this approach presently requires the surgical implantation of a device that places a permanent electrode in contact with the sacral nerve and possibly the tibial nerve. This invasive approach has the attendant risks and side effects of invasive surgery, including pain, swelling and inflammation, infection, tissue and nerve injury, plus required reoperation later to replace the device when its useful life ends.

In addition to use in controlling pelvic pain transmission and bladder function, (percutaneous) tibial nerve stimulation (PTNS) and other peripheral modulation can be used to modulate neuronal circuits to reduce localized chemokine expression and to suppress the entry of pathogenic cells. Tibial nerve stimulation affects the inflammatory reflex in which afferent vagus nerve signaling activated by inflammatory mediators, such as cytokines or pathogen-derived molecular signals, functionally culminates into efferent vagus nerve activation that dampens pro-inflammatory cytokine production. As outlined above, to accomplish this, the motor (efferent) signals in the vagus nerve activate the splenic nerve, which culminates in the release of acetylcholine (ACh) by a subset of T lymphocytes expressing choline acetyltransferase (ChAT). ACh interacts with the α7 nicotinic ACh receptor (α7nAChR) expressed by macrophages to inhibit cytokine production.

Monitoring of one or more physiological parameters may be used to determine when to initiate treatment, to confirm nerve modulation(s) through changes in specifically measured physiological parameters (or direct measurement of a change in nerve activity or neurologic impedance), and to confirm when to repeat treatment, in addition to other patient treatment decisions benefitted by real-time, dynamic monitoring. The monitoring of one or more physiological parameters on a real-time and dynamic basis may also advantageously be used to confirm both the successful nerve stimulation and to direct delivery and the related ongoing beneficial physiological result of treatment according to the invention. Monitoring one or more physiological parameters can allow biomarkers to be measured in real time (a significant advantage over the current reliance on sampling, testing and return of lab results, which can take hours for each sample) and to be measured repeatedly over time to establish trends for each biomarker monitored. As mentioned above, successful nerve stimulation involves activating the nerve in a manner that generates a signal from the nerve that can impact an aspect of the inflammatory reflex and/or reduce pain. The use of moving average statistical calculations and other interrelated statistical calculations based on monitoring one or more physiological parameters allows for real-time dynamic adjustment in the modulating of one or more nerves of the patient, in the administering of the therapeutic agent, or both during treatment. Real-time dynamic adjustments may be accomplished through use of artificial intelligence, neural networks, or other algorithm-based analyses to track and integrate the measure of one or more physiological parameters over time. A method of treatment according to the invention has the distinct advantage of combining real time monitoring and adaptive calculations based on machine learning and other artificial intelligence (AI) to identify and guide administration of therapeutic agents along with nerve modulation elements to address conditions more precisely than with current approaches. Using artificial intelligence, the experience and data from multiple patients may be used to optimize a method of treatment according to the invention and to also establish monitoring baselines to then be used to indicate successful nerve modulation across certain similar patient populations.

### Nerve Modulation

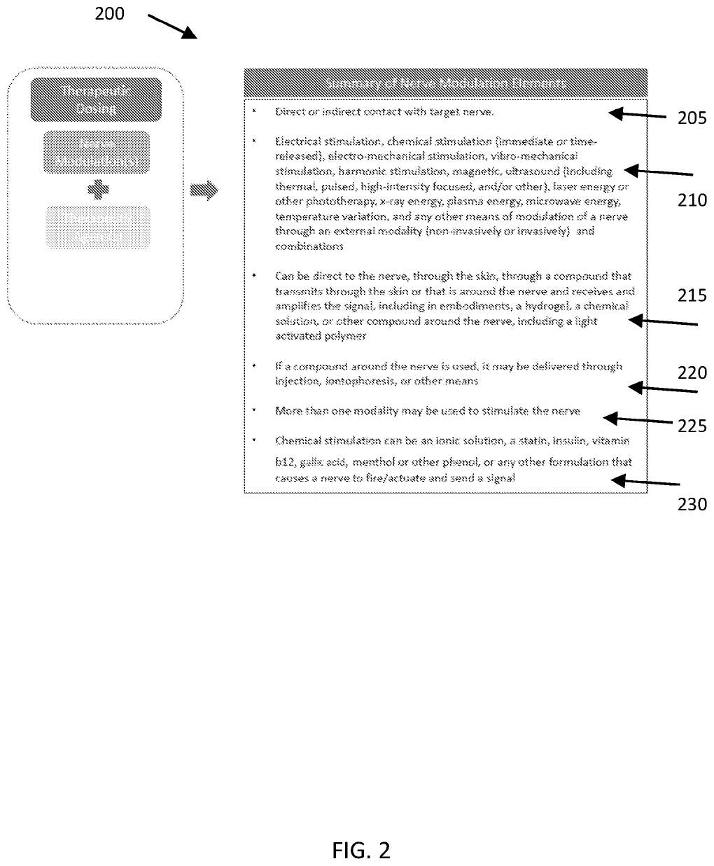

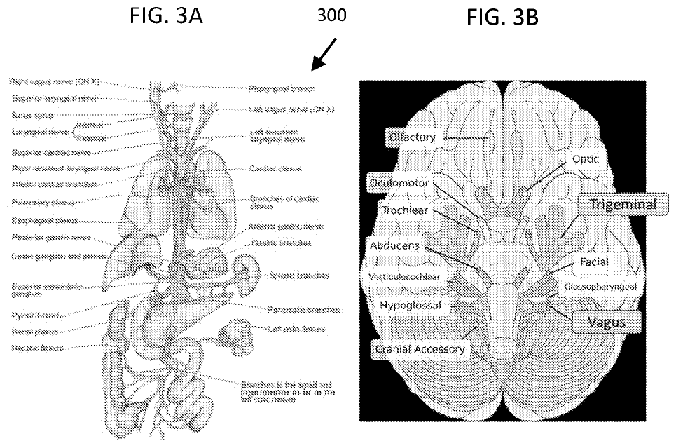

As shown in FIGS. 1-5, a method of treatment according to the invention includes, as part of its therapeutic dosing 200, a step of modulating one or more nerves of a patient. As block 205 shows, to modulate one or more nerves of a patent, a nerve modulation element is applied in a manner that stimulates one or more nerves in the nervous system to in turn communicate a response through the nervous system that results in an anti-inflammatory effect or other nerve-related response. As shown further in block 300, a modulation element or source delivers a signal to one or more nerves, such as for a vagus nerve, a trigeminal nerve, a splenic nerve, a cervical nerve, a tibial nerve, an alternate peripheral or central nerve, or a combination thereof, including, in embodiments, branches of one or more of these nerves. As outlined above, (percutaneous) tibial nerve stimulation (PTNS) and/or sacral nerve stimulation can be used to modulate neuronal circuits to address incontinence issues, as well as to reduce localized chemokine expression and to suppress the entry of pathogenic cells, as well. Nerve modulation in a method of the invention may be singular or have multiple objectives and/or beneficial effects. FIG. 2 summarizes elements of nerve modulation 200 according to the invention. FIGS. 3A and 3B depict the ANS nerve branches in the human body.

As shown in block 210, the nerve modulation may occur through one or more of a number of modalities, including, by way of example but without limitation, through the delivery of electrical stimulation to the nerve, chemical stimulation (immediate or time-released), electro-mechanical stimulation, vibro-mechanical stimulation, harmonic stimulation, magnetic, ultrasound (including thermal, pulsed, high-intensity focused, and/or other), laser energy or other phototherapy, x-ray energy, plasma energy, microwave energy, temperature variation, and any other means of modulation of a nerve through an external modality (non-invasively or invasively) or combinations of one or more of the foregoing forms of stimulation, as outlined in block 225.

As shown in block 215, in a method of treatment according to the invention, nerve modulation may be done using a signal delivered through an implanted device, through a device that is in contact with the skin, through a device that is distant from the skin, by means of a probe or other instrument that penetrates the skin, or by other means designed to stimulate a nerve. Nerve modulation may also be accomplished through implantable means in contact with or in close proximity to the nerve; through a temporary probe and/or needle in contact with the nerve or in proximity to the nerve; through a permanent conductive element; through a bioabsorbable conductive element, or through other forms of modulation with the modulation source internal or external to the patient. Nerve modulation may also be performed in contact with the skin of the patient (i.e., transcutaneously) or performed at a distance from the skin, but in a manner sufficient to modulate the nerve, or involving a combination of stimulation approaches. Delivery of the modulation signal may be through one or more electrodes, transducers, harmonic elements, ultrasonic elements, catheters, needles, vibrational probes, wires, nanoparticle, visible, ultraviolet, or infrared light from LEDs, lasers, or other sources, and/or application and/or induction of electromagnetic fields.

Nerve modulation according to the treatment methods of the invention may be delivered through a secured device on a targeted area to affect the nerve, such as using an attachment device, like the attachment device used for an ECG lead or similar monitoring element, and thereby positioned over, in proximity to, or in contact with a target nerve, such as the splenic nerve, a branch of the vagus nerve, the trigeminal nerve, tibial nerve, sacral nerve, or other nerve, or a combination thereof, which is able to produce a modulated nerve response, including without limitation, an anti-inflammatory response especially through the emission of anti-inflammatory T-cells and other anti-inflammatory cells from the spleen of the patient. A method of the invention may, as a separate step, also include administering an anti-inflammatory drug before, during or after either modulating at least one nerve or administering the therapeutic agent.

When modulation does not involve implantable means, the modulation element may be affixed directly to the skin or to a device affixed to the skin through a patch, adhesive material, microneedle fixation, expandable polymer or other securement device that may be separate or part of the nerve modulation element, in order to insure consistent, precise delivery, or the modulation element may be sized to fit within a specific body structure, such as the cymba concha or other aspect of the ear, including inside the ear, or other anatomy. In some embodiments, the modulation element may fit over the ear, or could be imbedded in a scarf or other adjustable element, or temporarily taped or otherwise affixed to the skin or in proximity to the skin, involve a finger cuff, arm cuff, wearable device, a probe, a wand, a hand-held device, or a combination thereof (including a patch). Similarly, other deliver locations may be utilized for targeted nerve application, including, for example, over or in proximity to the tibial nerve, sacral nerve, or splenic nerve or other peripheral nerves.

Nerve modulation may also be done through a natural opening of the body, such as through an eye drop of therapeutic drug/chemical to reach a branch of the trigeminal nerve or to stimulate one or more additional or other nerves. Alternatively, nerve modulation may be done by applying a transdermal patch containing a therapeutic drug/chemical to a patient's forehead, on or around behind the ear, and to areas of the shoulders, neck, and head innervated by cranial nerves or areas through which cranial or other targeted nerves, such as other peripheral nerves, traverse.

As shown in block 220, nerve modulation may occur through a bioabsorbable material or contact injected, delivered through iontophoresis or other transdermal delivery, or otherwise delivered around, in contact with, in proximity to or otherwise in a position in which a nerve modulating signal is delivered through the contact or agent, which in embodiments can be an electrode. The nerve modulation could also be through a hydrogel or other gel that is electrically conductive, or otherwise responds to an external signal and transmits that signal to the nerve in a manner that stimulates or modulates the nerve, or otherwise improves the signaling to the nerve, such as removing air between the nerve modulation element and the skin, nerve or other contact point with the patient.

As shown in block 230, a method of treatment according to the invention can include nerve modulation that is delivered chemically. The delivery may be through an immediate and/or a time-released delivery of the chemical to one or more nerves or to the tissues surrounding the nerve through injection. Alternatively, in some embodiments, the chemical delivery maybe infused, delivered orally, topically, or, in some embodiments, through a patch applied to the skin in proximity to one or more nerves with the ability to elude a chemical element that modulates the nerve, which may occur through microneedles, electrophoresis, iontophoresis or other transdermal delivery, or other delivery and/or elution means.

The chemical modulation may be through one or more drugs able to produce a modulation of a nerve, including, by way of example, but not limitation, targeting a muscarinic and/or b-adrenergic receptor; a vagomimetic drug such as acetylcholine, pyridostigmine, galantamine and/or choline, a statin, insulin, vitamin B12, saline, potassium, menthol, or other chemical elements able to cause a nerve to emit a signal.

Additionally, the nerve modulation may utilize one or more chemical elements to improve nerve response, such as the delivery of a formulation including Vitamin B12 to improve the nerve sheath response, and/or a gel to remove air space between the stimulation form and the skin covering a nerve, and/or a gel formulated to aid in the carrying of a stimulation means across the skin to the underlying nerve.

The dosing of the nerve signal may vary, depending on the physiology of the patient. For instance, the level of obesity in a patient can increase the physical distance between the surface of the skin and the neural target, thus changing the power requirements necessary to activate the intended nerve. Also, nerve fiber projections into the periphery, particularly cranial nerves innervating, for example, the meatus of the ear or the scalp or forehead, can vary between patients due to genetic variability or prior traumas.

In some example embodiments, if electrical energy is used for modulation of the nerve, the range of transmitted energy needed may vary by patient anatomy, and this amount of energy may be different than the energy actually reaching the nerve. In a preferred embodiment, between 0 and 100 milliamps may need to reach the nerve for modulation of the nerve. The delivery of electrical energy or other modulation elements, including magnetic, harmonic, ultrasound, laser or other phototherapy, chemical or other element may occur in a waveform, including forms with a pulse shape (e.g., sinusoidal, square, trapezoidal, or others). Various frequency ranges, amperage and voltage may be part of a preferred embodiment, depending on the specific patient, which may include but not be limited to a combination of frequencies (˜1-50 Hz), amperages (˜0.5-3.5 mA), voltages (˜5-60V), pulse widths (˜1-350 microsec), pulse shapes (e.g., sinusoidal, square, trapezoidal, etc.), charge balances (AC current, or DC current with a neutral electrode), interpulse intervals (for AC—the time between a positive pulse and a negative pulse), pulse trains (a number of pulses followed by a short “off” interval), duty cycles (amount of time “on” per hour), and intensities of therapy delivery (e.g., for example, the device may actuate for two minutes twice a day, or may actuate more frequently (for example, four minutes a day for two to four times a day, or every twenty-five seconds every ten minutes) or less frequently depending on the monitoring feedback indication, such that it could be turned on less frequently or more frequently depending on the patient's specific needs, ongoing disease condition, improvement or degradation in condition and other factors.

In some embodiments, the method of treatment according to the invention involves delivering one or more forms of modulation to the nervous system to create the production and release of anti-inflammatory cells including T-cells, monocytes/macrophages, and neutrophils, and anti-inflammatory cytokines including interleukins, such as for example but not limitation, (IL)-1 receptor antagonist, IL-4, IL-10, IL-11, and IL-13, which have the benefit of inhibiting pro-inflammatory processes elicited by exogenously delivered immunotherapies or endogenous disease pathologies.

Also, pain is frequently caused by pro-inflammatory cells and cytokines activating afferent pain pathways; thus, antagonizing pro-inflammatory responses can inhibit pain processes, as well as other actions such as activating the release of naturally-occurring endogenous opioids.

### Administration of Therapeutic Agent(s)

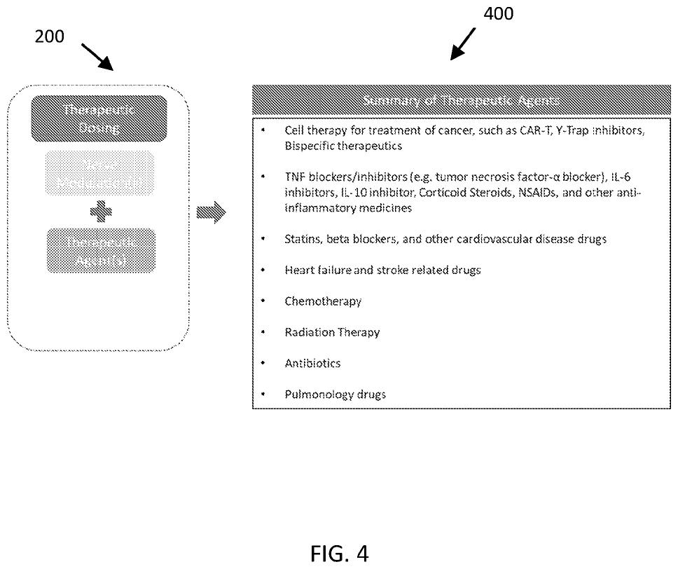

As shown in block 400 of FIG. 4, methods of treatment according to the invention include, as part of their therapeutic dosing, a step of administering a therapeutic agent to the patient in need thereof. In a method of the invention a particular therapeutic agent may be administered under its current therapeutic regimen or otherwise based on patient need. A method of the invention adds the step of modulating one more of the patient's nerves before, during or after administering a therapeutic agent to increase the therapeutic efficacy of the therapeutic agent being administered. Increasing therapeutic efficacy may involve increasing the desired direct therapeutic effect, decreasing side effects or both. Typically, a therapeutic agent is administered using the same dosages, amounts, and regimens in its prescribing information, although in an example method of the invention, lower dosages may be determined and used. FIG. 4 summarizes certain aspects of administering one or more therapeutic agents in methods of treatment according to the invention as part of the method's therapeutic dosing.

The invention also provides a related apparatus comprising a device to administer a therapeutic to the patient in need thereof. In some example embodiments, the invention provides a device to deliver a therapeutic to target a cytokine storm, such as a cytokine storm triggered by CAR-T, for example. In example embodiments of the invention, the devices used to administer a therapeutic include at least one of the group of microneedle patches, microparticle depots, transdermal patches, transdermal topical applicators, multiparticulate systems, nanoparticles, implantable devices (including controlled release implantable devices), polymer films, capsules (including pH-responsive capsules, injection devices, liquid delivery devices, intranasal delivery devices, infusion devices, and hydrogels. In some example embodiments of the invention, the devices deliver compounds as secondary therapeutics, such as to target the cytokine storm that CAR-T triggers, while in some example embodiments, the devices deliver both a “primary” therapeutic (e.g., CAR-T) and a “secondary therapeutic” at the same time and/or with the same device.

As example embodiments of the invention, methods of treatment involve treating a patient's immune system by modulating one or more nerves of the patient prior to administering an immune system modifying agent, while administering an immune system modifying agent, or after administering an immune system modifying agent. In other embodiments, a method of treatment according to the invention involves treating a patient by modulating one or more nerves of the patient prior to, while, or after administering therapeutic agents known to either cause inflammatory conditions, or administering therapeutic agents which are used to treat inflammation related conditions, such as for example, rheumatoid arthritis, lupus, irritable bowel syndrome, sepsis/septic shock/acute respiratory distress syndrome (including that caused by novel pathogens such as SARS-COV-2), diabetic complications, or other autoimmune diseases, infectious diseases (such as HIV and AIDs) or cardiovascular diseases; neuroinflammatory conditions including stroke, traumatic brain injury/concussion, post-traumatic stress disorder, and seizure disorders; and diseases of autonomic dysfunction including fibromyalgia, opioid withdrawal syndrome, insomnia, chronic pain, headaches, incontinence, hypertension, stress/anxiety, autism/attention deficit hyperactivity disorder, bleeding conditions, postural orthostatic tachycardia syndrome, or postoperative ileus. Examples of therapeutic agents which may be administered to a patient in need thereof according to a method of the invention include, but are not limited to, an immunotherapy drug, a drug to treat an autoimmune condition, an anti-inflammatory drug, a blood clotting factor (such as Factor VIII), a steroid, a gasdermin-D inhibitor (such as Disulfiram), an antibody, antiviral (or antiviral combination) or other agent to treat an infectious disease, a Chimeric Antigen Receptor T-Cell therapy, Chimeric Antigen Receptor Macrophage therapy, or other cell-based immune-oncology therapeutic, or a gene-based therapy, as outlined in FIG. 4.

A method of treatment according to the invention may also be used as a separate, standalone therapy to reduce cytokine levels and other indicators of inflammation in a patient, as well as to treat other conditions, such as asthma, rheumatoid arthritis, lupus, irritable bowel syndrome, sepsis/septic shock/acute respiratory distress syndrome (including that caused by novel pathogens such as SARS-COV-2), diabetic complications, or other autoimmune diseases, infectious or cardiovascular diseases; neuroinflammatory conditions including stroke, traumatic brain injury/concussion, post-traumatic stress disorder, and seizure disorders).

A method of treatment according to the invention involves treating a patient's immune system by modulating one or more nerves of the patient prior to or in connection with administering a chemotherapy or other immune system modifying agent to suppress the patient's immune system prior to administration of CAR-T, CAR-M or other immune-oncology therapy.

### Monitoring Physiological Parameters

An example method of treatment according to the invention includes an optional step of monitoring one or more physiological parameters of the patient undergoing treatment as shown in block 100 in FIG. 1. The need or desirability of monitoring depends on the particular treatment. Monitoring, particularly real-time and dynamic monitoring of one or more of the patient's physiological parameters permits adaptive, statistical-based analysis and learning/adjustments involving measurement of one or more physiological parameters. Monitoring can confirm the successful nerve stimulation(s) and, on a real-time and dynamic basis, can confirm both successful nerve stimulation and ongoing beneficial physiological results. Using moving average statistical calculations and other interrelated statistical calculations based on multiple physiological parameters, the systems and methods in accordance with the invention can confirm the nerve modulation techniques produce a physiological response that is intended and beneficial to the patient. In an example method of treatment according to the invention, monitoring one or more physiological parameters of the patient confirms the modulation of at least one nerve of the patient and the administration of the therapeutic to the patient. The nerve modulation and/or the therapeutic administration may be adjusted in response to a monitored physical parameter.

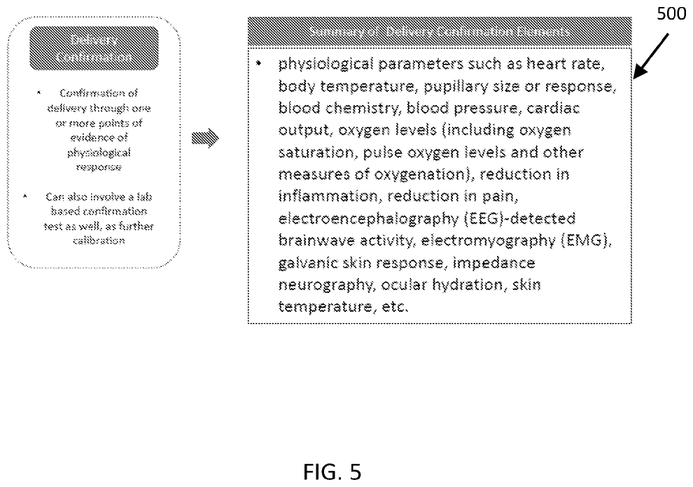

A monitoring step may then be used to confirm a therapeutic dosing aspect of the methods, which may include confirming the modulation of at least one nerve of the patient and/or confirming the administration of the therapeutic to the patient. In an example method of treatment according to the invention, monitoring one or more physiological parameters of the patient on monitors on a real-time and dynamic basis informs the nerve modulation and/or the administration of the therapeutic agent. Monitoring one or more physiological parameters of the patient on a real-time and dynamic basis allows comparison to a baseline of one more physiological parameters of the patient and may establish when to begin or to repeat modulation of a nerve, to begin or to repeat administration of a therapeutic agent or both. For example, as shown in block 500 in FIG. 5, monitoring a physiological parameter to confirm nerve modulation and/or therapeutic agent effects, may involve monitoring one or more physiological parameters such as, but not limited to, heart rate, body temperature, pupillary size or response, blood chemistry, blood pressure, cardiac output, oxygen levels (including oxygen saturation, pulse oxygen levels and other measures of oxygenation), reduction in inflammation, reduction in pain, electroencephalography (EEG)-detected brainwave activity, electromyography (EMG), galvanic skin response, impedance neurography, ocular hydration, skin temperature, and the like.

A method of treatment which includes monitoring at least one physiological parameter of the patient has at least three uses and advantages: 1) Monitoring engagement of the target nerve by the neuromodulation strategy; 2) Monitoring for the emergence of a cytokine response syndrome with a subset of the measured physiological parameters; and 3) Monitoring for improvement to the cytokine response syndrome following application of the neuromodulation strategy.

A method of treatment according to the invention may also monitor physiological parameters including biomarkers of the immune/inflammation, cardiopulmonary, gastrointestinal, and central and autonomic nervous systems. These biomarkers serve to confirm target engagement of the neuromodulation intervention, monitor therapeutic efficacy, and assess patient responses to both immunomodulation therapy (to detect development of cytokine response syndrome) and neuromodulation therapy (to inhibit cytokine response syndrome). Each of these biomarkers represents a single dimension by which these responses can be assessed, and each can be monitored over at least two additional dimensions-time and magnitude of change from baseline, among others.

An apparatus to practice the invention by monitoring pupillary response may use a device for covering one or more eyes of the patient in connection with measuring pupillary response in order to prevent outside stimuli, such as light or other elements, from adversely impacting the measurement of the pupil of one or more eyes for pupillary response prior to an attempt to modulation a nerve, in connection with an attempt at nerve modulation, and following nerve modulation. The device may be a patch, eyeglasses, a hood, a funnel shaped cover, or other means of limiting or excluding external light and other outside stimuli. A pupillary response device includes a measurement element, which may be a camera or other means to capture before and after views of the pupil(s) in real time or otherwise. The camera may be part of a smart phone that can then transmit the image into a measurement program; a smart phone with an app that can measure, a tablet; a computer with an internal or external camera or a stand-alone device capable of capturing and/or measuring a change in the pupils.

In addition, a monitoring step of one or more physiological parameters of the patient has the important benefit of enabling personalized approaches to patient care in the context of delivering nerve modulation and a therapeutic agent. Certain patients may not be candidates for effective monitoring in this context without access to more than one parameter and real-time dynamic information with key information obtained, analyzed, and compared over a period of time and related activities. For example, with some forms of nerve stimulation or modulation, such as modulating the vagus nerve, a change in heart rate variability can indicate the vagus nerve has been successfully modulated. However, without a patient-specific baseline, other factors can also cause a change in heart rate variability, so unless one can compare the heart rate to a patient-specific baseline, this form of nerve modulation confirmation may not be reliable. Additionally, certain patients may have heart conditions, such as heart arrhythmias, that do not support reliable measurement of heart rate variability due to this form of heart disease. Other patients may have had this form of heart disease treated with a pacemaker or implantable defibrillators, which also prevent reliable measurement of heart rate variability due to the intentional effects of the pacemaker or implantable defibrillator.

Utilizing a single physiological parameter or an alternative physiological parameter may introduce other issues unless the physiological parameter is combined with other physiological parameters and/or coupled with additional physiological parameters. For example, pupillary response can change in connection with successful vagus nerve and/or trigeminal and/or tibial and/or other peripheral nerve modulation, however the reliability and usefulness of this parameter also requires an effective baseline and can lose its effectiveness as a monitoring parameter in a number of circumstances. For example, if the patient has experienced a concussion or other neurological trauma, pupillary response may be modified such that it loses its effectiveness as a monitoring parameter. Additionally, certain drugs and other substances can impact pupillary response that adversely impacts its usability in this context as a precise monitoring parameter to indicate successful nerve modulation. Even when the patient's physical condition allows for the use of pupillary response as an indicator of successful nerve modulation, the excitation of the pupil to determine if nerve modulation is successful requires an absence of other stimuli that can excite the pupil or an accurate indication of a clear pupillary response related to the attempted modulation of the nerve will not be obtained. Stray forms of light can all cause the pupil to respond without there being an actual nerve modulation. Also, pupillary response can also vary patient to patient depending on the level of health, age, and potential diseases the patient is combatting, whether the patient has forms of ocular disease (such as, for example, cataracts), so developing a pupillary response baseline without distortion from extraneous stimuli is important to successfully confirm effective nerve modulation.

In a method of treatment according to the invention, then, monitoring one or more physiological parameters of a single patient undergoing treatment may occur across dozens of dimensions. These data may also be integrated with physiological parameter data from other patients using an artificial intelligence and/or neural network platform, or other algorithmic means, to analyze data and present trends to the healthcare provider. Thus, the step of monitoring one or more physiological parameters in a method of treatment according to the invention may include, along with or separate from the confirming step, a step of integrating physiological parameter data of the patient being treated with physiological parameter data from other patients treated with a method of treatment according to the invention. Depending upon the physiological data used, the patients may have been treated with the same nerve modulation technique, a different nerve modulation technique, administered the same therapeutic agent, or administered a different therapeutic agent. In other words, the patient population may have undergone the same therapeutic dosing or a different therapeutic dosing using a method of treatment according to the invention.

### EXAMPLES

Animal experiments were performed to demonstrate that peripheral nerves can be activated via delivery of chemical agents targeted to interact with nerve receptors to create signaling in the cervical vagus nerve. Delivery was performed topically and transdermally.

Chemical compounds were applied to the vagus nerve in the neck or to trigeminal nerve fibers in the forehead or to peripheral nerves of several rats, while simultaneously recording vagus nerve activity in the neck. The trigeminal nerve is part of the greater vagal network, and the compounds were applied to the trigeminal via intradermal injection to mimic transdermal iontophoresis.

### Summary of Experiments:

This testing demonstrates that targeted compounds activate peripheral nerves via agonists of chemoreceptors or somatosensory receptors resulting in signaling in the cervical vagus nerve. These compounds demonstrate chemical nerve activation with similar effects as electrical stimulation of the vagus nerve, but through chemical agents engaging nerve sensors, but with effects like electrical stimulation of the vagus nerve, which has known therapeutic effect in multiple disease states including epilepsy, depression, opioid withdrawal syndrome, headache disorders, and stroke rehabilitation (neuroplasticity), inflammation reduction and others.

### Approach and Methods:

Adult male Lewis rats (350-500 g) were anesthetized with isoflurane via nosecone, and then maintained on continuous infusion of alfaxan. Rats were implanted with femoral venous and arterial catheters for delivery of anesthetic and monitoring of heart rate and blood pressure, respectively. The cervical vagus nerve was isolated from the left carotid artery and suspend on bipolar platinum electrodes connected to BioPac recording hardware. Agonists of transient receptor potential cation channel subfamily V (TrpV) or subfamily M (TrpM) in various concentrations were applied to the exposed cervical vagus nerve, or were injected intradermally on the forehead in the vicinity of trigeminal nerve fiber endings. Cervical vagus nerve activity was recorded continuously, and compound applications were timestamped in the BioPac Acknowledge software. After the experiments were completed, data acquired with Acknowledge software were converted to MatLab file types using Python. MatLab was then used to analyze 60-second segments of recorded vagus nerve activity, from −70 sec to −10 sec prior to agonist application (pre-treatment, “PRE”) and from +10 sec to +70 after agonist application (post-treatment, “POST”). The root mean square (RMS) was calculated for each 60-second segment, and nerve activity spikes were defined as voltages>3×RMS. Spike activity was computed as spikes/second+/−standard error of the mean (SEM). Comparisons of spike counts were made between POST and PRE conditions using a two-tailed paired T test; p<0.05 was deemed significant.

### Example 1. Effects of Capsaicin on Cervical Vagus Nerve

Baseline recordings of spontaneous vagus nerve signaling revealed average spike counts of 3.6+/−1.3 spikes/second. Capsaicin is a known agonist of TRPV1, a chemoreceptor with known expression on cranial nerve fibers including trigeminal nerves. Application of low (10 nM) concentrations of capsaicin directly on the cervical vagus nerve increased the average spike count (7.6+/−0.7 spikes/second, p=0.03). Application of high (100 nM) concentrations of capsaicin directly on the cervical vagus nerve led to a further increase in average spike count (14.5+/−0.7 spikes/second, p=0.03). Intradermal application of high (100 nM) concentrations of capsaicin increased the cervical vagus nerve average spike count to 13.5+/−0.5 spikes/second (p=0.008).

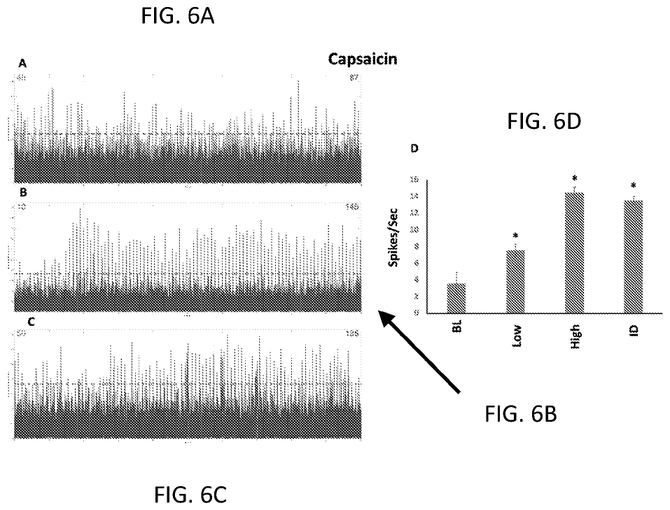

As shown in FIGS. 6A-6C, representative 10-second cervical vagus nerve recordings were obtained at baseline (green, panels A-C), or after cervical applications of capsaicin at low (red, A) or high (red, B) concentrations, or intradermal application at high (red, C) concentrations. FIG. 6D shows a histogram representation of cervical vagus nerve activity at baseline (BL); after low (Low) or high (High) concentrations of capsaicin were applied to the cervical vagus nerve; and after intradermal application of capsaicin (ID) in the forehead near trigeminal nerve endings (data presented as mean+/−SEM; *=p<0.05).

TRPV1 is a chemoreceptor expressed on multiple tissues including lung, gastrointestinal tract, joints, skin, and the trigeminal nerve. Capsaicin, an active component of chili peppers, is an agonist of TRPV1. Application of capsaicin directly to the cervical vagus nerve induced increased vagal nerve activity as detected by spike count analysis of electroneurograms recorded from the cervical vagus nerve. Intradermal application of capsaicin to trigeminal dermatomes on the forehead also activated nerve firing of the main cervical vagus nerve, suggesting that chemoreceptor-mediated afferent signaling to the brain can activate efferent activity of the vagus nerve into the viscera.

By extension, other agonists of TRPV1, including, but not limited to, oxytocin, ketamine, palvanil, olvanil, resiniferatoxin, anandamide, 12-hydroxyeicosatetranenoic acid, N-arachidonoyldopamine, piperine and zingerone, have similar activity to capsaicin. Like capsaicin, these compounds also activate TRPV1 or open the TRPV1 ion pore, and thus activate nerve firing. Other molecules that modulate TRPV1 activity to induce neuronal firing include, for further example, calcium calmodulin, bradykinin, serotonin, histamine, or prostaglandins,

### Example 2. Effects of GSK1016790A on Cervical Vagus Nerve

Baseline recordings of spontaneous vagus nerve signaling revealed average spike counts of 7.5+/−1.6 spikes/second. GSK1016790A is a known agonist of TRPV4, a chemoreceptor with known expression on cranial nerve fibers including trigeminal nerves. Application of low (10 nM) or high (100 nM) concentrations of GSK1016790A directly on the cervical vagus nerve had no effect on spike count (data not shown). Intradermal application of low (10 nM) concentrations of GSK1016790A did not increase cervical vagus nerve average spike count (7.0+/−0.5 spikes/second (p=0.829). Intradermal application of high (100 nM) concentrations of GSK1016790A increased the cervical vagus nerve average spike count to 13.8+/−1.0 spikes/second (p=0.015).

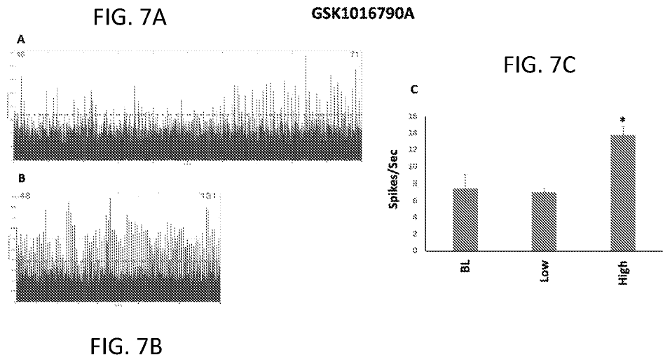

As shown in FIGS. 7A-7B, representative 10-second cervical vagus nerve recordings were obtained at baseline (green, panels A-B), or after intradermal applications of GSK1016790A at low (red, A) or high (red, B) concentrations. FIG. 6C shows a histogram representation of cervical vagus nerve activity at baseline (BL); and after intradermal application of low (Low) or high (High) concentrations of GSK1016790A in the forehead near trigeminal nerve endings (data presented as mean+/−SEM; *=p<0.05).

TRPV4 is a chemoreceptor expressed on multiple tissues including lung, gastrointestinal tract, joints, skin, and the trigeminal nerve. GSK1016790A is a potent and selective agonist of TRPV4. Application of GSK1016790A directly to the cervical vagus nerve induced increased vagal nerve activity as detected by spike count analysis of electroneurograms recorded from the cervical vagus nerve. Intradermal application of GSK1016790A to trigeminal dermatomes on the forehead also activated nerve firing of the main cervical vagus nerve, suggesting that chemoreceptor-mediated afferent signaling to the brain can activate efferent activity of the vagus nerve into the viscera.

By extension, other agonists of TRPV4, including but not limited to 4-phorbol 12,13-didecanoate (4PDD) and its derivatives, the epoxyeicosatrienoic acids 5,6-EET and 8,9-EET, bisandrographolide A, RN-1747, and derivatives of GSK1016790A, have similar activity to capsaicin. Like GSK1016790A, these compounds also activate TRPV4 or open the TRPV4 ion pore, and thus activate nerve firing.

### Example 3. Effects of WS-12 on Cervical Vagus Nerve

Baseline recordings of spontaneous vagus nerve signaling revealed average spike counts of 4.3+/−0.7 spikes/second. WS-12 is a known agonist of TRPM8, a chemoreceptor with known expression on autonomic and somatosensory nerve fibers in the lungs, gastrointestinal tract and skin. Application of low (1.2 μM) concentrations of capsaicin directly on the cervical vagus nerve increased the average spike count to 11.2+/−0.6 spikes/second (p=0.02). Application of high (12 μM) concentrations of capsaicin directly on the cervical vagus nerve increased the average spike count to 17.7+/−0.8 spikes/second (p=0.007). Intradermal application of high (12 μM) concentrations of capsaicin increased the cervical vagus nerve average spike count to 9.6+/−0.5 spikes/second (p=0.03).

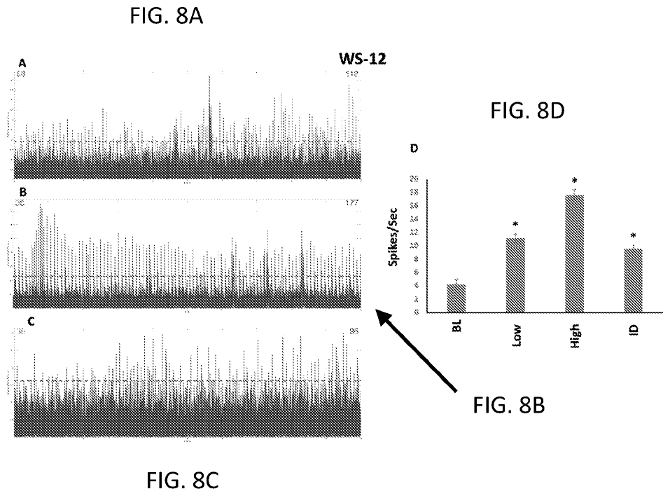

As shown in FIGS. 8A-8C, representative 10-second cervical vagus nerve recordings were obtained at baseline (green, panels A-C), or after cervical applications of WS-12 at low (red, A) or high (red, B) concentrations, or intradermal application at high (red, C) concentrations. FIG. 8D shows a histogram representation of cervical vagus nerve activity at baseline (BL); after low (Low) or high (High) concentrations of WS-12 applied to the cervical vagus nerve; and after intradermal application of WS-12 (ID) in the forehead near trigeminal nerve endings (data presented as mean+/−SEM; *=p<0.05).

TRPM8 is a molecular transducer of cold sematosensation, which is expressed on sensory neurons. Certain compounds, including menthol and its derivatives, are also capable of activating TRPM8; of the menthol derivatives, WS-12 is one of the most selective. Application of WS-12 directly to the cervical vagus nerve induced increased vagal nerve activity as detected by spike count analysis of electroneurograms recorded from the cervical vagus nerve. Intradermal application of WS-12 to trigeminal dermatomes on the forehead also activated nerve firing of the main cervical vagus nerve. These observations indicate that TRPM8-mediated afferent signaling to the brain can activate efferent activity of the vagus nerve into the viscera.

By extension, other agonists of TRPM8, including but not limited to menthol, icillin, and dialkylphosphorylalkanes such as 1-diisopropylphosphorylnonane (cryosim-3), I-menthol, 1-diisopropylphosphoryl-pentane, 1-diisopropylphosphoryl-hexane, 1-diisopropylphosphoryl-heptane, 1-diisopropylphosphoryl-octane, 1-diisopropylphosphoryl-nonane, 1-di-sec-butylphosphoryl-4, 1-di-sec-butylphosphoryl-pentane, 1-di-sec-butylphosphoryl-hexane, 1-di-sec-butylphosphoryl-heptane, 1-di-sec-butylphosphoryl-octane, 1-diisobutylphosphoryl-pentane, 1-di-sec-butylphosphoryl-3-methyl-butane have similar activity to WS-12. Like WS-12, these compounds activate TRPM8 or open the TRPM8 ion pore, and thus activate nerve firing.

By extension, agonists of other neuronal chemoreceptors have similar nerve-activating properties when applied intra- or transdermally as capsaicin, GSK1016790A, and WS-12. Other chemoreceptors include, but are not limited to, other members of the transient receptor potential channel family, including TRPC, TRPV, TRPVL, TRPM, TRPS, TRPN, TRPA, TRPP and TRPML; cannabinoid receptors CB1, CB2, GPR18, GPR55/CB3, GPR119, and the PPAR family of receptors; and opioid receptors delta, kappa, mu, nociception receptor, and zeta. The compounds are known to activate peripheral nerves including cranial and tibial nerves, and thus intra- or trans-dermal application of agonists of these receptors have similar neuromodulatory properties.

### CONCLUSIONS

The systems, devices, and methods in accordance with the invention use targeted compounds to activate peripheral nerves including via agonists of chemoreceptors or somatosensory receptors resulting in signaling in the cervical vagus nerve. These compounds demonstrate chemical nerve activation with of the vagus nerve, but through chemical agents engaging nerve sensors, but with effects like electrical stimulation of the vagus nerve, which has known therapeutic effect in multiple disease states including epilepsy, depression, opioid withdrawal syndrome, headache disorders, stroke rehabilitation (neuroplasticity), inflammation reduction, incontinence, and others. Chemical stimulation of the vagus nerve includes similar therapeutic effects as electrical vagus nerve stimulation in several additional indications including rheumatoid arthritis, inflammatory bowel disease, acute and chronic COVID-19, post-traumatic stress disorder, acute stroke, and traumatic brain injury.

1. A method of treatment comprising the steps of:
modulating one or more nerves of a patient,
administering a therapeutic agent to the patient in need thereof, and
optionally monitoring one or more physiological parameters of the patient.

2. A method of treatment of claim 1, wherein the step of modulating one or more nerves of the patient includes the patient's vagus nerve, trigeminal nerve, splenic nerve, cervical nerve, tibial nerve, or a combination thereof.

3. A method of treatment according to claim 1, wherein the step of administering a therapeutic agent to a patient in need thereof administers a therapeutic agent for the treatment of at least one of the group of inflammation, pain, cancer, cardiovascular disease, asthma, rheumatoid arthritis, lupus, irritable bowel syndrome, sepsis/septic shock/acute respiratory distress syndrome (including that caused by novel pathogens such as SARS-CoV-2), diabetic complications, or other autoimmune diseases, infectious diseases; bleeding, neuroinflammatory conditions including stroke, traumatic brain injury/concussion, post-traumatic stress disorder, seizure disorders, incontinence, and postoperative ileus.

4. A method of treatment according to claim 1, wherein the method of treatment includes the step of monitoring one or more physiological parameters of the patient.

5. A method of treatment of claim 4, wherein the monitoring step establishes one or more baselines of one or more physiological parameters of the patient.

6. A method of treatment according to claim 4, wherein the method includes the step of monitoring one or more physiological parameters of the patient to confirm the nerve modulation.

7. A method of treatment according to claim 4, wherein the method includes the step of monitoring one or more physiological parameters of the patient on monitors on a real-time and dynamic basis to inform the nerve modulation, the administration of the therapeutic agent, or both.

8. A method of treatment according to claim 5, wherein the method includes the step of monitoring one or more physiological parameters of the patient on a real-time dynamic basis compared to a baseline of one more physiological parameters of the patient to establish when to begin or repeat modulation of a nerve, to begin or repeat administration of a therapeutic agent, or both.

9. A method of treatment according to claim 1, wherein the step of modulating one or more nerves of the patient occurs before the step of administering a therapeutic agent to a patient in need thereof.

10. A method of treatment according to claim 1, wherein the step of modulating one or more nerves of the patient occurs during the step of administering a therapeutic agent to a patient in need thereof.

11. A method of treatment according to claim 1, wherein the step of modulating one or more nerves of the patient occurs after the step of administering a therapeutic agent to a patient in need thereof.

12. A method of treatment according to claim 1, wherein the method of treatment includes the step of monitoring one or more physiological parameters of the patient to confirm the modulation of at least one nerve of the patient, the administration of the therapeutic to the patient, or both.

13. A method of treatment according to claim 1, wherein the method of treatment includes the step of monitoring one or more physiological parameters of the patient integrates physiological parameter data across a patient population.

14. A method of treatment according to claim 1, wherein the method of treatment includes the step of modulating one or more nerves of a patient, the step of administering a therapeutic agent to the patient in need thereof, or both steps are adjusted in response to a monitored physical parameter.

15. A method of treatment according to claim 1, further comprising the step of administering an anti-inflammatory drug to the patient.

16. An apparatus comprising a device to modulate one or more nerves of a patient before, during or after administration of a therapeutic to the patient in need thereof, a device to deliver the therapeutic to the patient in need thereof, and a device to monitor one or more physiological parameters of the patient.
